(19) United States
(12) Patent Application Publication
(10) Pub. No.: US 2005/0125019 A1
(43) Pub. Date: Jun. 9, 2005

(54) Lancet device and method

(76) Inventors: Paul Kudrna, Spring Grove, IL (US); Brian Schwartz, Lake in the Hills, IL (US); Mark Schwartz, Wauconda, IL (US)

Correspondence Address: Wallenstein Wagner & Rockey, Ltd., 53rd Floor, 311 S. Wacker Drive, Chicago, IL, 60606-6622, US

(21) Appl. No.: 10/728,741

(22) Filed: Dec. 5, 2003

(51) Int. Cl. A61B 17/14; A61B 17/32

(52) U.S. Cl. 606/182; 606/181

(57) ABSTRACT

A lancet device (10) having an internal magnet (20) and the forces emanating therefrom driving and retracting a collar (30) and a lancet (60). The device has a steady state condition (SS) wherein the device is at rest and in equilibrium. The device can be armed and activated to puncture. Once puncturing has occurred the device returns to a steady state condition.

A lancet method involves positioning both a magnet (20) and a member (30) capable of being affected thereby with a lancet (60) in communications with one of either the magnet or the member, positioning either the member (30) or the magnet (20) to an armed position wherein the magnetic forces from the magnetic affect the member, and releasing the one of either the member or the magnet from the armed position permitting movement between the member and magnet by at least, in part, the magnetic forces, resulting in the movement of the lancet from a withdrawn position to the piercing position.

### TECHNICAL FIELD

The present invention generally relates to lancets, and more particularly, to a magnetically driven lancet.

### BACKGROUND OF THE INVENTION

In treating several medical conditions or injuries, samples of blood must be taken and tested. For example, to treat diabetes with insulin, blood sugar levels must be checked, or monitored, regularly. As a result, blood must be drawn from the individual requiring the check and tested by any one of a number of known methods. One such method is placing a small amount of blood on a test strip and having the test strip read in a specially designed meter.

Many advances have reduced the amount or volume of blood needed for medical tests. However, for many, drawing blood can be frequent. In addition, for many medical conditions or injuries, individuals draw their own blood without the need of professional medical personnel. The result of this has been the development of personal lancing devices for individuals to draw their own blood. Such devices cover a spectrum from simple, e.g., a finger pricking device (needle, pin, lancet), to quite complicated (mechanical or electronic devices).

Central to any device is the amount of pain resulting from its use. Many traits or characteristics have been identified as increasing or decreasing the pain associated with such devices. For example, vibration. Problems needing to be addressed in devices include reducing or eliminating vibration. Side-to-side vibration (transverse oscillation) of a lancet's tip while moving to puncture skin causes an irregular puncture, causing unnecessary pain and discomfort to a user. Repeated bouncing into and out of the skin occurs upon insertion of the lancet into the puncture or lancing site. This back-and-forth motion into and out of the skin is a pogo-stick effect or a form of longitudinal oscillation. Finally, speed of the lancing device into and out of the lancing site affects pain greatly. A slower speed of puncture and withdrawal from a site causes more pain.

Over the years both companies and individuals have strived to improve upon lancets. Goals include facilitating use of the lancets, reducing the pain caused by the lancets, reusability of lancet, ease of use, reducing size, reducing noise associated their use, etc.

### SUMMARY OF THE INVENTION

The present invention is an improvement upon existing lancets. It is relatively easy to use and load with lancets presently on the market. It is quiet in use. The lancet tip moves quickly (into the skin and from the skin) and without vibration or oscillation to generate a quick and straight piercing in the user, both aspects reducing pain associated with the lancing process. The piercing depth can also be easily controlled or adjusted. In short, the device of the present invention can be customized to a particular users desires and repeated over and over so that each piercing is substantially the same.

Other advantages and aspects of the present invention will become apparent upon reading the following description of the drawings and the detailed description of the invention.

### BRIEF DESCRIPTION OF THE DRAWINGS

In the accompanying drawings forming part of the specification, and in which like numerals are employed to designate like parts throughout the same,

FIGS. 1 and 2 are front perspective views of the device of the present invention:

FIG. 3 is an exploded view of the lancing device of the present invention;

FIGS. 4-9 are side elevation schematic views of the device showing the steps to arming the device;

FIG. 10 is a perspective front view of the dial adjuster and follower;

FIG. 11 is a cross-sectional view of the device showing the dial adjuster and follower;

FIG. 12 is a further sectional view of the dial adjuster, follower and lancet;

FIG. 13 is a close-up of portion of FIG. 12 circled;

FIGS. 14-18 show the different lancet positions available by using the dial adjuster;

FIGS. 19-24 are side sectional views of the device showing the different positions of components during operation of the lancet within the device after arming and during puncturing;

FIGS. 25 and 26 are density plots of the magnet within the device and the magnetic fields generated thereby; and,

FIG. 27 is a schematic representation of the magnet, the magnetic fields and the collar in representative positions.

### DETAILED DESCRIPTION OF THE INVENTION

While this invention is susceptible of embodiments in many different forms, there is shown in the drawings and will herein be described in detail, preferred embodiments of the invention with the understanding the present disclosure is to be considered as an exemplification of the principles of the invention and is not intended to limit the broad aspect of the invention to the embodiments illustrated.

The present invention uses the full, complete, 360 degrees, magnetic field of a magnet to project the tip of a lancet outwardly from the device from a safe, steady state position to a puncturing position and to retract the lancet back into the protected, steady state position. The complete use of the magnetic field reduces oscillation, or movement, of the tip in directions other than the traveling direction of the tip. This greatly increases the efficiency of the lancet and reduces pain potentially caused thereby.

The Components of the Lancing Device 10

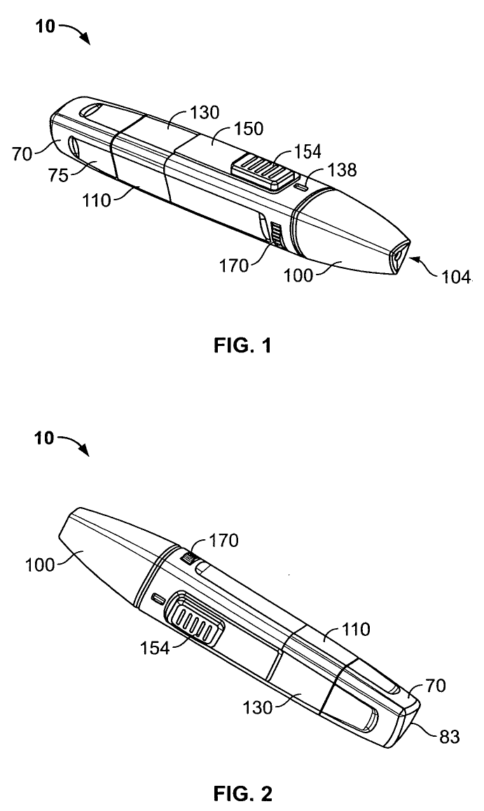

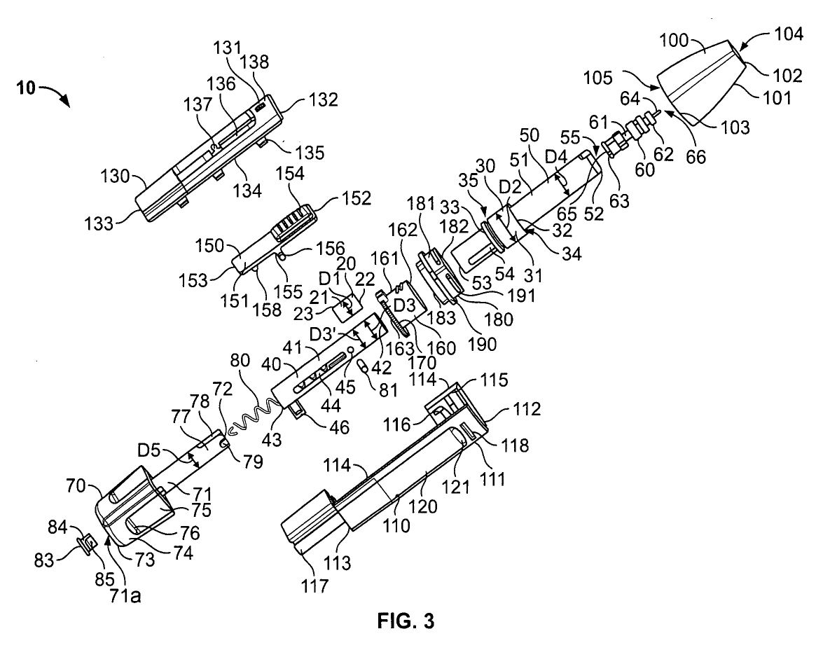

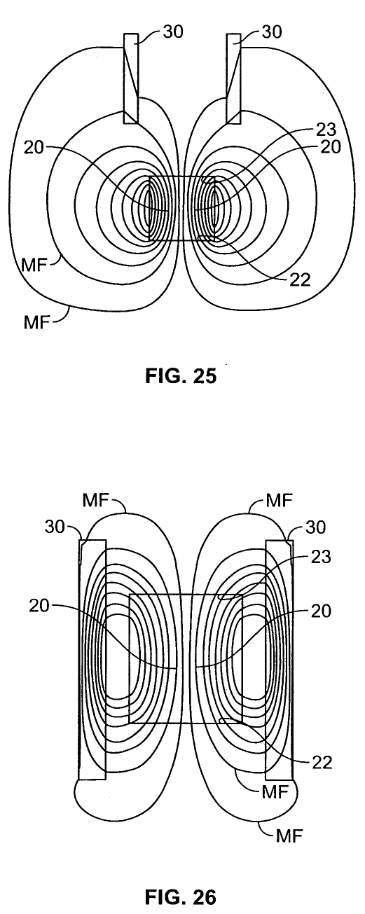

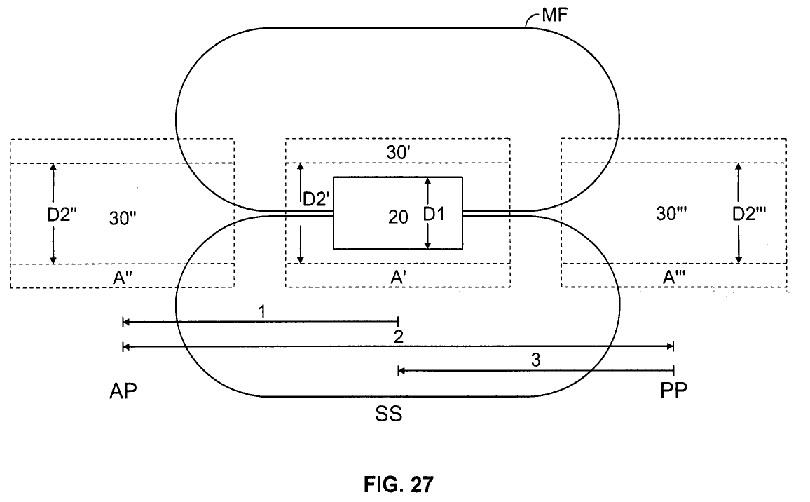

FIG. 1 is an exploded view of the primary components of the device, generally designated by reference number 10. Central to the lancing device 10 is a magnetic element 20 and a collar 30. The magnetic element 20 is preferably a solid cylindrical magnet having an outer surface 21, a front end 22 and a rear end 23. The magnet 10 further has a magnet diameter D1. The magnetic field(s) generated by the magnet 10 is shown in FIGS. 25 and 26 and schematically in FIG. 27.

Generally, the cylindrical magnet 20 has two poles; one pole is charged North (N) on one flat end 23 and the other pole is charged South (S) on the opposing flat end 22. This results in magnet field curved lines of flux being generated between both of the poles and around the entire circumference of the cylindrical magnet.

As shown in FIG. 27, the relationship between the magnet 20 and collar 30 are shown in three positions—Steady State (SS), Armed Position (AP) and Piercing Position (PP). As discussed in detail below, the magnet 20 is stationary and during arming, the collar 30 is moved along path 1 from a steady state position to an armed position. When the device 10 is activated, the collar moves along path 2 from the armed position to the piercing position and further along path 3 from the piercing position back to the steady state position. A representative point “A” on the collar is shown in the three positions.

The collar 30 is preferably an annulet, tubular having an outer surface 31, a front end 32, a rear end 33 and an inner surface 34. The collar 30 is composed, at least in part, or has associated therewith material capable of being affected by the magnetic force(s) emanating from the magnet 20. Thus, the collar 30 can be drawn or pulled towards the magnet 20 when the magnetic forces are attractive between the magnet and the collar. In the device of the present invention, the forces used are always attractive. Thus, the magnet is always drawing metal to it. Specifically referring to FIG. 27, the attractive forces of the magnet are drawing the collar towards the magnet (path 2) and the momentum of the moving collar causes the collar to move past the magnet. Once at the piercing position, the collar will stop and the drawing/attractive forces of the magnet will pull the magnet back to the steady state or equilibrium position. Suitable materials for the collar 30 are iron, steel (plated or stainless) or any ferrous metal. Preferably, the collar is made of stainless steel or any form of plated, non-corrosive steel. The annular inner surface 34 has a collar diameter D2 slightly greater than the outer diameter D1 of the magnet 20. As a result, the magnet 20 can pass longitudinally through the collar 30. In addition, the outer surface 34 of the collar has an circumferential channel 35 therein spaced from the rear end 33 of the collar 30 for cooperating with holding means to hold the collar in the arming position.

The magnet 20 is fixedly secured, preferably by force fitting or an adhesive, to an inner shaft 40 and the collar is fixedly secured, also by an adhesive, around a separate outer shaft 50. Both the inner shaft 40 and the outer shaft 50 are generally tubular having front ends 42,52 and rear ends 43,53 with the inner shaft having an outer diameter D3 and the outer shaft having an inner diameter D4. The outer diameter D3 of the inner shaft 40 is less than the inner diameter D4 of the outer shaft 50. This sizing allows the inner shaft 40 to move longitudinally relative to the outer shaft 50. In short, the inner shaft 40 can ride and travel within the outer shaft 50.

The inner shaft 40 also has an elongated longitudinal slot 44 therein, opposed holes 45 and a generally radial flange 46 projecting therefrom which aligns and retains the shaft 40 to the first housing component 110. The outer shaft 50 similarly has an elongated longitudinal slot 54 therein. The outer shaft 50 also includes a receptacle section or portion 55 at the front end 52 thereof.

The lancet 60 generally has a centrally located body 61 with a front end 62 and a rear end 63, a tip 64 projecting longitudinally and outwardly from the front end and a key 65 at the rear end. The outermost end of the tip 64, spaced from the body 61, is the point 66 of the tip for puncturing. The receptacle 55 in the outer shaft 50 is contoured and configured to receive and securely seat and hold the keyed end 65 of the lancet 60. The lancet 60 is molded plastic, save the metal tip 64, and can thus be customized. In addition, in the preferred embodiment, the receptacle 55 at the front end 52 of the outer shaft 50 is constructed so as to accept, seat, secure and hold commercially available lancets manufactured by others in the industry, such as ComforTouch™ by Lowen Mumford, Soft Touch™ by Roche Diagnostics and Ultra-Fine™ II by Becton, Dickinson & Company (BD). Such customized construction of the lancet and receptacle portion of the shaft is easily understood by those in the art of molding and molding techniques. It is recognized that instead of customizing the front end 52 of the outer shaft 50 to be a receptacle 55, one can optionally employ a separate insert (not shown) to attach to or in the front end of the outer shaft to act as the receptacle, and keyed for mating with the keyed end 65 of the lancet 60. Thus, one can easily and regularly remove one disposable lancet 60 after use and replace it with a new lancet for future use.

Mentioned above, the outer shaft 50 moves (slides) relative to the inner shaft 40. Discussed in more detail below, the lancet 60 secured to the outer shaft 50 moves relative to the inner shaft 40. Specifically, the lancet 60 is moveable between a withdrawn position (wherein it is protected within the housing (discussed below)) and a piercing position (discussed below) wherein the tip 64 is fully extended, for the particular setting, to pierce the individual user with the point 66. This movement by the lancet is driven the pulling caused by the magnetic forces of the magnet 20 and their interrelation with the collar 30. See discussion relating to FIG. 27.

An arming member 70 is provided to arm, or move, the lancet 60, collar 40, outer sleeve 50 and inner sleeve 40 into position for use (piercing). This arming member 70 includes a front end 72 and rear end 73, along with a gripping portion 74 at the rear end 73. A centrally located aperture 71a is provided in the rear end 73 and gripping portion 74. The arming member 70 is preferably made of molded plastic and may be double molded or coated with a high friction material so as to prevent the user's fingers from slipping from the member when the user pulls it rearwardly to arm the device. To facilitate gripping and this pulling action by the user (“arming”), a plurality of radially spaced annular recesses 75 and marginal walls 76 are constructed in the gripping portion 74.

The mentioned aperture 71a opens up into tubular portion 71 projecting forwardly from the gripping portion 74. This tubular portion 71 has an outer diameter D5, slightly less than the inner diameter D3′ of the inner shaft 40 to let the tubular portion slide within and relative to the inner shaft. A plurality of tangs 77, formed between slots 78 are formed at the distal or front end 72 of the member 70. A guide, or projection 79, also extends radially outward from the end 72. The tangs 72 provide a spring action ensuring the tubular section can be inserted into the inner shaft and slid longitudinally therein. They 79 also ensure the guide 79 is biased radially outwardly. This guide 79 is configured so as to project through and move relative to (when the arming member 70 is moved) both the longitudinal slot 44 formed in the inner shaft 40 and the longitudinal slot 54 formed in the outer shaft 50.

In assembling the device 10, the front end 72 of the arming member 70 is compressed and inserted into the rear end 43 of the inner shaft 40 until the guide 79 protrudes through the slot 44. An extension spring 80 is then put into the inner shaft 40, the front end of the spring being held in the shaft by a spring-retainer 81 inserted into one end of the inner shaft 40. The spring-retainer 81 is fixedly secured by the extension spring applying a pulling force upon it, forcing a seat of the spring-retainer upon a mating ledge within the inner shaft 40. Alternately the spring-retainer can be additionally further retained (by sonic welding, adhesives or other conventional methods) to the shaft 40. The rear end of the spring 80 is held in the inner shaft 40 and arming member 70 by a passageway 85 provided in the body portion 84 of an end cap 83. The end cap 83 seats within the centrally located aperture 71a in the rear end 73 of the gripping portion 74 of the arming member 70. It is also understood that the end cap 83 can be molded integral to the tubular member 71 such that they are one component.

This assembly ensures the arming member stays connected to the inner shaft 40.

Protecting the tubular portion 71 of the arming member 70, the inner shaft 40 and magnet 20, the outer shaft 50 and collar 30 and the dial adjuster 160 and follower 180 (discussed below) is an outer, concentric housing comprised of a first housing component 110 and a second housing component 130. These components 110,130 mate with one another to generally protect the just noted internal workings of the device 10. Each housing component 110,130 has an outer surface 111,131, a front end 112,132 and a rear end 113,133 and abutting edges 114,134 for mating with the other housing component. In particular, the first housing component 110 has a ridge 115 running along the inner surface thereof along the abutting edge while the second housing component 130 has a plurality of barbs 135 projecting downwardly therefrom. The barbs 135 of the second housing 130 grip and hold the ridge 115 of the first housing member when the two 110,130 are put together or, more appropriately, snap fitted together. Adhesive, sonic welding, or compression tapered fitting can also be used to retain both housing components 110,130 together along their respective abutting edges 114,135. As with the arming member 70, the first housing 110 also includes one or more annular recesses 120 and walls 121 to facilitate gripping of the device 10. The first housing member 110 further has an extension 117 extending rearwardly from rear end 113. This extension fits between the tubular portion 71 and the gripping portion 74 of the arming member 70 when the device 10 is assembled and the arming member 70 is not extended. This housing component further includes a window 118 in the outer surface 111 to permit a user to rotate a dial 170 projecting therefrom and to view the setting or other indicia 171 (discussed below relating to adjusting the piercing position and the point 66 of the tip 64 of the lancet 60). Internal ribs 116 are also molded within the first housing component 110 to space the component from the internal mechanics or to hold the respective parts in their respective positions relative to one another.

The second housing member 130 has a central annular opening 136 therein, along with a two downwardly extending clips 137 to accommodate and hold a button switch 150. The opening or window 138 is for viewing the numbers (171) printed or molded on the dial adjuster 160. Internal ribs (not shown in FIG. 1) are also molded within the second housing component 130 to space the component from the internal mechanics or to hold the respective parts in their respective positions relative to one another. While the housing is shown as two pieces, it is appreciated that it can, if desired, be a single piece.

The button switch is 150 is coupled to the second housing member 130 and is seated within the annular opening 136. The button 150 has an outer surface 151, front end 152, rear end 153 and a touch pad 154 constructed/molded adjacent the front end. Centrally located are two downwardly extending flanges 155, each with an outwardly projecting protuberances 156. The protuberances 156 are snap fitted into the clips 137 of the housing 130. This coupling of the housing 130 and button 150 together lets the button rotate relative to the housing. In short, the button 150 can pivot about the clips 137/protuberances 156 like a rocker-switch or teeter-totter.

Adjacent the rear end 153 of the button switch 150, below the portion rearwardly of the flanges 155/protuberances 156, a transverse crest 158 is provided. This crest 158 is a ridge or rib for mating with the circumferential channel 35 of the collar 30. The crest 158 sits within the channel 35 when the device 10 is armed. Specifically, the collar 30 is released (or free to move or translate longitudinally) when the touch pad 154 is touched and the crest is pivoted away from and out of the channel 35. It should be understood that the button my also incorporate conventional and well known means (springs, etc.) to bias the crest 158 towards and into the collar 30 and the channel 35.

The cap 100 is juxtaposed or adjacent to the front ends 112,132 of the housings 110,130. In the preferred embodiment, the cap 100 is not connected directly to the housing 110,130, rather the cap 100 is connected to a follower 180 disposed between the housing 110,130 and the cap. The cap also has a front end 102, a rear end 103 and an outer surface 101. It 100 is pyramidal or conical, tapered towards the front end 102. Both the front end 102 and rear end 103 of the cap 100 have openings 104,105. The cap 100 protects the lancet 60 from dust and debris and protects the user, as well as others, from inadvertently contacting the tip 64 and point 66 of the lancet. The cap 100 can easily be removed from the housing 110,130 to remove and replace the lancet 60 and then reconnected to the housing.

To facilitate the removing and replacing of the cap 100, the cap has one or more internal, circumferential, annular grooves 107 spaced from the rear end 103 to snap fit or engage one or more circumferential crowns 191 constructed on the external surface of the follower 180. (See FIG. 13).

In use, the user puts his/her skin against the front opening 104 of the cap 100 and activates the device 10. Once activated, the tip 64 of the lancet is propelled from within the cap 100 to outside the cap and the point 66 moves from a withdrawn position (within the cap/housing) to it “piercing position,” that position furthest from the front end 102 of the cap to lance the user. After reaching the just noted piercing point, the tip 64 and point 66 withdraw to a position back within the cap 100/and housing 110,130.

The dial adjuster 160 works in conjunction with the follower 180 to adjust the cap 100 relative to the housing 110,130 and the lancet 60 and for selectively setting the positioning of the just mentioned piercing position. The follower 180 abuts, but is not secured to, the outer surface 51 of the outer shaft 50 generally adjacent the front end 52 and receptacle 55 of the shaft (when the device is in its steady state). The front end 182 of the follower 180 acts as a guide for the shaft 50 and the lancet 60 and reduces oscillation of the lancet when it is activated, projecting forward in the device and piercing. Specifically, the follower 180 contacts the outer shaft 50 a full 360 degrees; this minimizes lancet 60 oscillation during longitudinal motion of the lancet, translating into less pain during puncture.

The dial adjuster 160 is also not attached to the outer surface 51 of the outer shaft 50. The dial adjuster 160 is screw fitted into the follower 180 and is maintained in position by the housing 110,130. Consequently, the dial adjuster 160 is rotatable relative to the follower 180 to increase the distance or gap between the housing 110,130 and the cap 100, or more particularly, between the point 66 of the lancet 60 and the front end 102 and front opening 104 of the cap. In short, rotation of the dial adjuster 160 translates to longitudinal motion of the follower 180 and cap 100 relative to the outer shaft 50, lancet 60 and lancet point 66.

It should be noted the dial adjuster 160 does not translate relative to the housing 110,130, but the follower 180 along with the cap 100 do move (longitudinally) relative to the housing. The dial adjuster 160, and hence the follower 180, are held in place relative to the housing 110,130 by the dial 170 of the adjuster 160 projecting through one or more circumferential, annular windows 118 in the outer surface 111,131 of the housing components 110,130.]

Adjusting the Piercing Position of the Lancet

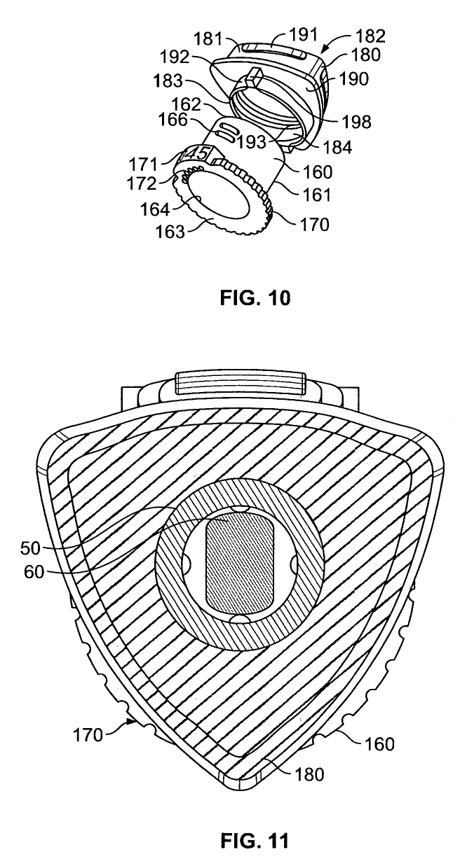

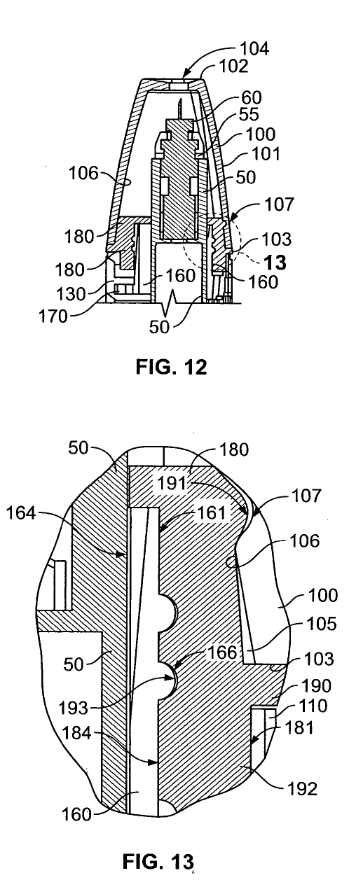

The details of the interrelationship between the dial adjuster 160 and follower 180 are shown in FIGS. 10-13. Specifically, as with the other components, the follower 180, which cooperates with the dial adjuster 160, has a front end 182, a rear end 183, an outer surface 181 and an inner surface 184. Similarly, the dial adjuster 160 has a front end 162, a rear end 163, an outer surface 161 and an inner surface 164. Both the follower 180 and the dial adjuster 160 are tubular annulets and have threading. Specifically, the dial adjuster 160 has circumferential external helical threads 166, or portions thereof, on the outer surface 161 adapted to cooperate with circumferential inner helical troughs 193 (internal threads) in the inner surface 184 of the follower 160. The outer diameter of the dial adjuster is slightly less than the inner diameter of the follower. As a result, the external threats 166 mate with the internal threads 193 to permit rotation of the dial adjuster 160 relative to the follower 180, or of the follower relative to the dial adjuster.

The outer surface 181 of the front end 182 of the follower 180 is contoured to match the rear opening 105 at the rear end 103 of the cap 100. Spaced from the front end 182 of the follower are elongated crowns 191 for cooperating with one or more internal, elongated, circumferential grooves 107 formed in the inner surface 106 adjacent the rear end 103 of the cap 100. A radial flange 190 on the follower 180 acts as a stop and abutment for the rear end 103 of the cap 100. A tubular section 192 projects rearwardly of the flange for receiving the dial adjuster 160. The tubular section 192 also includes an indicator 198 formed thereon behind the radial flange 190.

A radial dial 170 is constructed, or molded, at the rear end 163 of the dial adjuster 180. Like a flange, this dial extends outwardly from the outer surface 181 of the adjuster 180. The outermost surface of the dial 170 is serrated to facilitate its gripping or fingertip rotation. The dial also includes a plurality of spaced apart radial peaks 172 and an indicator portion 171 having indicia thereon, such as number 1, 2, 3, 4 and 5, to facilitate adjustment and of the dial adjuster. The peaks 172 coincide with the indicia so as to optionally hold the dial 170 in a desired position.

In particular, peaks 172 are provided to act as ratcheting or friction detent points which provide tactile feedback to the user turning the dial adjuster. The dial 170 of the adjuster 160 projects through one or more windows 118 in the outer surface 111,131 of the housing components 110,130. The windows 118 also permits one to view the indicia on the indicator portion 171 of the adjuster 160.

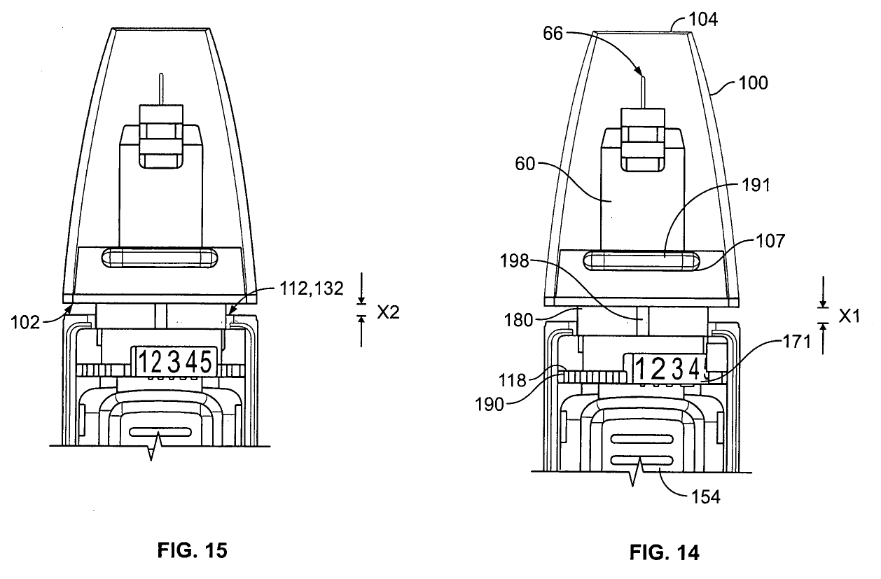

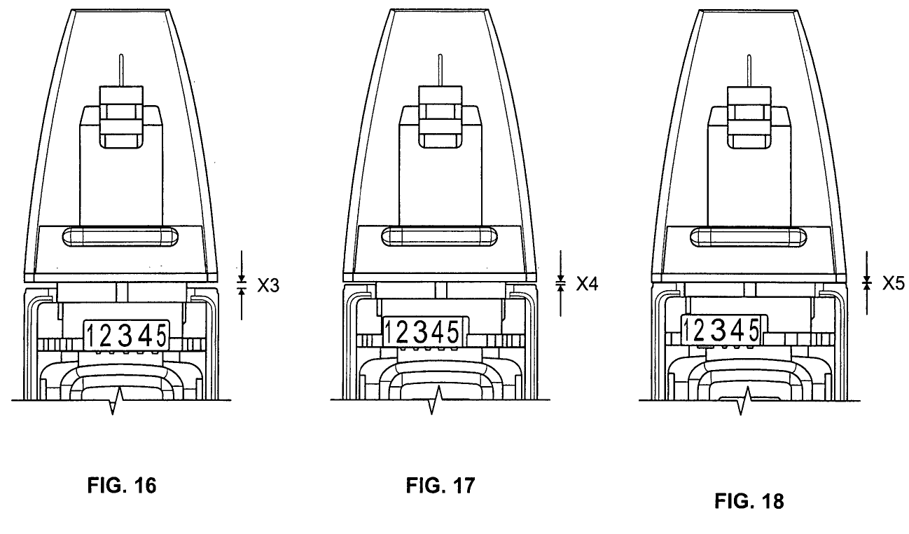

FIGS. 14-18 show the adjustment of the lancet device 10. Noted previously, the dial adjuster 160 is rotated relative to the follower 180 to increase the distance or gap between the housing 110,130 and the cap 100. As the dial 170 is rotated, the height of the follower 180, and hence the cap 100, changes. More particularly, this gap—shown as X1-X5 in FIGS. 14-18, is the specific distance between the rear end 102 of the cap 100 and the front end 112,132 of the housing 110,130. As the dial 170 is rotated, this gap (X1-X5) increases or decreases. Rotation of the dial 171 and dial adjuster 160 translates to longitudinal movement of the follower 180 and attached cap 100. As this gap increases (X5 to X1), the distance between front end 102 of the cap 100 and point 66 of the lancet 160. Thus, the largest setting of the dial, indicated by the number 5 and gap distance X5 in FIG. 18, provides the shortest distance between the cap end 102 and lancet point 66 when the lancet is in the withdrawn position. When activated, this small gap distance X5 will translate to an increased, or furthest-most penetration, of the point 66 outside the cap end. In other terms, the piercing position, noted previously will become the greatest distance from the cap, resulting in the deepest or greatest penetration of the activated lancet 60. Conversely, the smallest setting of the dial, indicated by the number 1 and gap distance X1 in FIG. 14, provides the largest or longest distance between the cap end 102 and lancet point 66 when the lancet is in the withdrawn position. When activated, this large gap distance X1 will translate to a decreased, or closest-most penetration, of the point outside the cap end. In other terms, the piercing position will become the smallest distance from the cap, resulting in the shallowest or smallest penetration of the activated lancet 60. Intermediate gap distances X2, X3 and X4 are shown in FIGS. 15-17, respectively.

Arming the Lancing Device 10

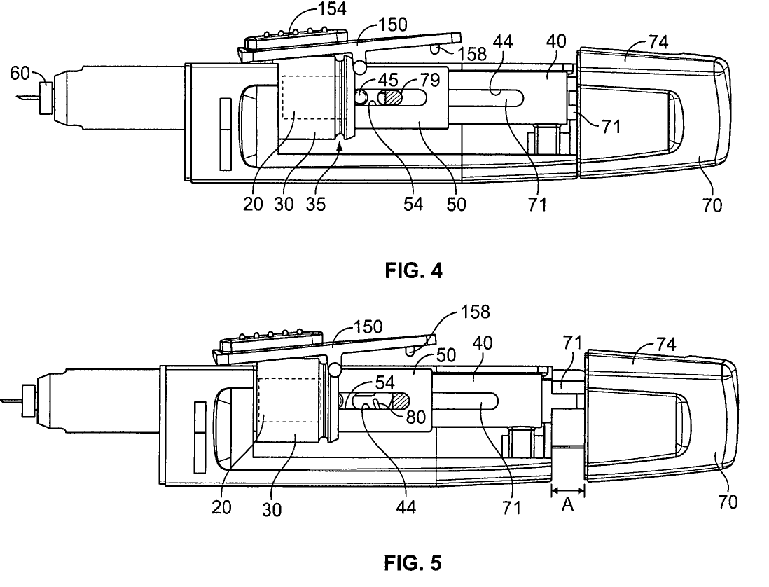

The arming of the device 10 is shown in FIGS. 4-9. The process generally involves going from a “steady state” condition to a “fully armed” condition. In the steady state, or neutral condition or position, the lancet will not translate or project outwardly from the cap. In the fully armed condition/position, the device is ready for activation. When activated, the lancet translates longitudinally within the cap and projects outwardly from the cap to pierce or puncture the user. Immediately upon piercing, the lancet retracts and withdraws into the cap back to the steady state condition.

In FIG. 4, the “steady state” condition is depicted. In the steady state position, the following conditions occur or are observed:
a) The arming member 70 is not extended.
b) The collar 30 encircles the magnet 20 and the 360 degree magnetic forces emanating from the magnet hold the collar in place.
c) The inner shaft 40 and outer shaft 50 are oriented so that the collar is situated around the magnet.
d) The gripping portion 74 of the arming member 70 abuts the housing 110,130 and the tubular portion 71 is substantially within the inner shaft 40. In addition, the extension 117 of the first housing member 110 is between the tubular portion 71 and the gripping portion 74 of the arming member 70.
e) The guide 79 at the distal end of the tubular portion 71 of the arming member 70 projects through both the longitudinal slot 44 formed in the inner shaft 40 and the longitudinal slot 54 formed in the outer shaft 50.
f) The button switch 150 is in the “disengaged position” wherein the crest 158 is disengaged from the channel 35 in the collar 30. Consequently, the outer shaft and attached collar are free to physically slide longitudinally relative to the inner shaft and magnet.

In the next figure, FIG. 5, the “one-quarter extended” condition is depicted. In the one-quarter extended position, the following conditions occur or are observed:
a) The arming member 70 is pulled or drawn about one-quarter the distance from the housing 110,130. As a result, the arming member is extended about one-quarter the total distance it is capable of being drawn from the housing.
b) The drawing of the arming member causes the guide 79 at the distal end of the tubular portion 71 of the arming member 70 projecting through both the longitudinal slot 44 formed in the inner shaft 40 and the longitudinal slot 54 formed in the outer shaft 50 to move rearwardly in both slots. The guide 79 contacts the rearward end of the outer shaft's slot 54. Once the outer rearward end of the outer shaft's slot 54 has been contacted, any further drawing of the arming member 70 also draws the outer shaft 50 and collar 30. In this figure, the outer shaft has moved rearwardly slightly.
c) The just noted movement of the outer shaft 50 longitudinally and rearwardly results in the same movement of the collar 30 from the steady state condition with the magnet 20.
d) The inner shaft 40 and outer shaft 50 have been moved relative to one another. The collar 30 is no longer centered around the magnet 20; rather, the collar is slightly rearward of the magnet. The magnet forces radiating from the magnet are pulling the collar towards the magnet and to the front of the device, opposite the rearward motion caused by the pulling action on the arming member. Consequently, a user feels a slight resistance when drawing the arming member from the housing.
e) The gripping portion 74 of the arming member 70 is spaced distance A from the housing 110,130 and the tubular portion 71 is partially withdrawn from and extending rearwardly and outside the inner shaft 40.
f) The button switch 150 is in the “disengaged position” wherein the crest 158 is disengaged from the channel 35 in the collar 30. The outer shaft and attached collar are free to physically slide longitudinally relative to the inner shaft and magnet.

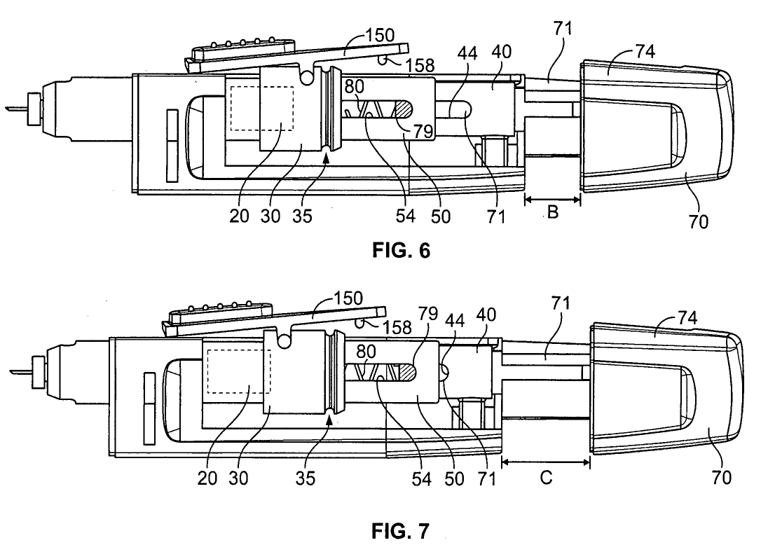

In the next figure, FIG. 6, the “one-half extended” condition is represented. In the one-half extended position, the following conditions occur or are observed:
a) The arming member 70 is pulled about half the distance from the housing 110,130. As a result the arming member is extended about one-half the total distance it is capable of being pulled from the housing.
b) The drawing of the arming member causes the guide 79 at the distal end of the tubular portion 71 of the arming member 70 projecting through both the longitudinal slot 44 formed in the inner shaft 40 and the longitudinal slot 54 formed in the outer shaft 50 to move rearwardly in the inner slot. The guide 79 having contacted the rearward end of the outer shaft's slot 54, now draws the outer shaft and collar 30 with the drawing of the arming member 70. In this figure, the outer shaft has moved rearwardly.
c) The just noted movement of the outer shaft 50 longitudinally and rearwardly results in the same movement of the collar 30 from the steady state condition with the magnet 20.
d) The inner shaft 40 and outer shaft 50 have been moved relative to one another. The collar 30 is further rearward of the magnet. The magnet forces flowing from the magnet continue to pull the collar towards the magnet and to the front of the device, opposite the rearward motion caused by the pulling action on the arming member. Consequently, a user continues to feel a slight resistance when drawing the arming member from the housing.
e) The gripping portion 74 of the arming member 70 is spaced distance B from the housing 110,130 and the tubular portion 71 is partially withdrawn from and extending rearwardly and outside the inner shaft 40.
f) The button switch 150 is in the “disengaged position” wherein the crest 158 is disengaged from the channel 35 in the collar 30. The outer shaft and attached collar are free to physically slide longitudinally relative to the inner shaft and magnet.

In FIG. 7, the “three-quarter extended” condition is represented. In the three-quarter extended position, the following conditions are present:
a) The arming member 70 is pulled about three-quarters the distance from the housing 110,130.
b) The drawing of the arming member 70 causes the guide 79 to move rearwardly in the inner slot 44. Because the guide 79 is contacting the rearward end of the outer shaft's slot 54, drawing the arming member 70 also draws the outer shaft 50 and collar 30. In this figure, the outer shaft has moved further rearwardly.
c) The just noted movement of the outer shaft 50 longitudinally and rearwardly results in the same movement of the collar 30 further from the steady state condition with the magnet 20.
d) The inner shaft 40 and outer shaft 50 have been moved relative to one another. The collar 30 is further rearward of the magnet. The magnet forces flowing from the magnet continue to pull the collar towards the magnet and to the front of the device, opposite the rearward motion caused by the pulling action on the arming member. Consequently, a user continues to feel a slight resistance when drawing the arming member from the housing.
e) The gripping portion 74 of the arming member 70 is spaced distance C from the housing 110,130 and the tubular portion 71 is substantially withdrawn from and extending rearwardly and outside the inner shaft 40.
f) The button switch 150 is in the “disengaged position” wherein the crest 158 is disengaged from the channel 35 in the collar 30. The outer shaft and attached collar are free to physically slide longitudinally relative to the inner shaft and magnet.

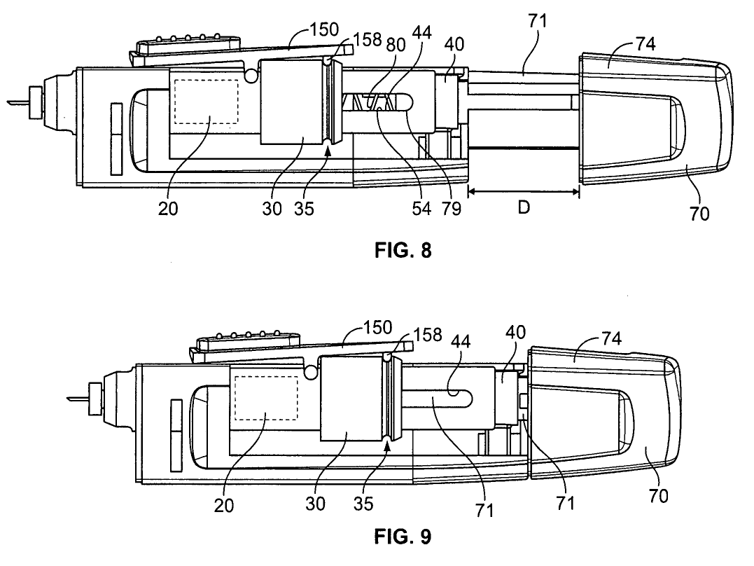

In FIG. 8 the “fully extended” condition is represented. In the fully extended position, the following are observed:
a) The arming member 70 is pulled completely from the housing 110,130 except for the furthest front portion of the tubular member 71 and guide 79.
b) The drawing of the arming member 70 causes the guide 79 to move rearwardly in the inner slot 44. The drawing of the guide 79 contacting the rearward end of the outer shaft's slot 54 draws the outer shaft 50 and collar 30. In this figure, the outer shaft has moved further rearwardly so that the crest 158 in the button switch 150 is aligned with the channel 35 in the collar and the crest may be seated (by the user or by mechanical biasing means, such as a biasing spring) within the channel.
c) The outer shaft's 50 movement longitudinally and rearwardly results in corresponding movement of the collar 30 from the steady state condition with the magnet 20.
d) The inner shaft 40 and outer shaft 50 have been moved relative to one another. The collar 30 is now completely rearward of the magnet. The magnet forces from the magnet act to pull the collar towards the magnet and to the front of the device, opposite the rearward motion caused by the pulling action on the arming member.
e) The gripping portion 74 of the arming member 70 is spaced distance D from the housing 110,130 and the tubular portion 71 is substantially withdrawn from and extending rearwardly and outside the inner shaft 40.
f) The button switch 150 can now be engaged (the “engaged position”) from the “disengaged position” because the crest 158 is aligned with the channel 35 in the collar 30. Once the button switch is engaged, the outer shaft and attached collar are basically held physically in position and prevented from sliding longitudinally relative to the inner shaft and magnet.

In FIG. 9 the “fully extended” condition is again represented. However, once the collar 30 is engaged and held in position by the button switch, the user can push the gripping member 70 back to the housing 110,130. Except for the movement of the guide 79 and tubular member 71 of the gripping member 70, the just noted fully extended conditions are still in place.

The magnet forces emanating from the magnet 20 are, in essence, trying to pull the collar 30 towards the magnet and to the front of the device, but the collar is held in position by the button 150. The device 10 is now armed and ready for use.

Operation of the Lancing Device 10

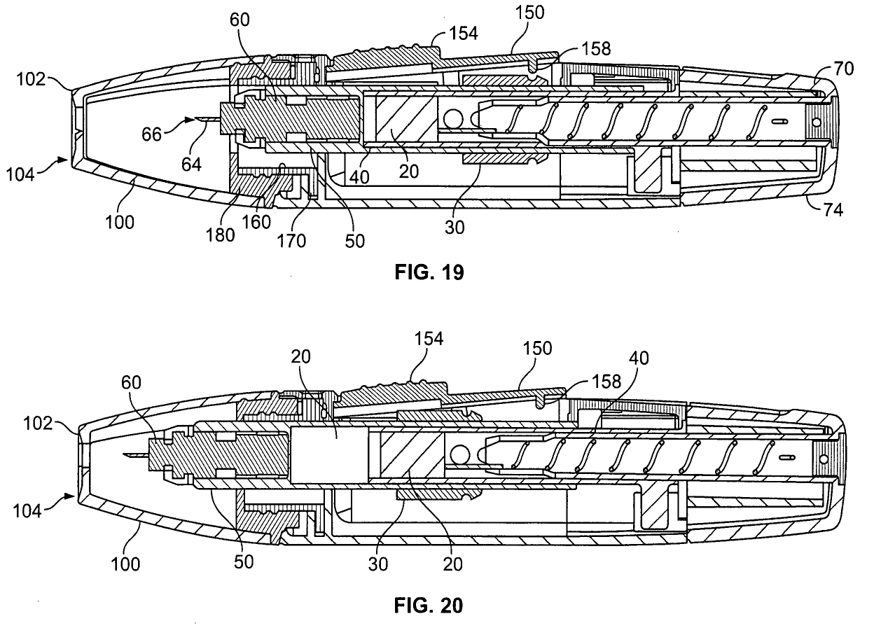

The activation of the lancing device 10 is shown in FIGS. 19-24. Specifically, FIG. 19 shows the same situation as existing in FIG. 9, the completely armed position or condition. This is the also the initial point of activation or release. At this juncture:
a) The collar 30, now rearward of the magnet 20, is engaged (the engaged position) and held in position by the button switch 150. The crest 158 is aligned and seated with the channel 35 in the collar 30. The outer shaft 50 and attached collar 30 are basically held and locked physically and prevented from sliding longitudinally relative to the inner shaft 40 and magnet. The gripping member 70 abuts, or is adjacent, the back of the housing 110,130.
b) The magnetic forces from the magnet 20 radiate to attract the collar 20 towards the magnet and to the front 102 of the device 10.
c) The lancet 60 and its respective tip 64 and point 66 are in a totally or complete withdrawn position, protected completely by the cap 100.

The touch pad 154 on the button switch 150 is touched, activating the device 10.

FIG. 20 shows the device 10 and lancet 60 just after activation at an intermediate point of action. As shown:
a) The magnetic forces from the magnet 20 radiate to pull the just released collar 30 towards the magnet and to the front 102 of the device 10. The magnet drives the just released collar, along with the outer shaft 50 and lancet 60, to the front of the device.
b) The crest 158 is no longer seated with the channel 35 in the collar 30, allowing relative motion between the inner shaft 40 and outer shaft 50.
c) The lancet 60 and its respective tip 64 and point 66 are still withdrawn and completely protected by the cap 100 but moving quickly towards the cap's opening 104.

FIG. 21 shows the device 10 and lancet 60 after the intermediate point of action and at the initial point of puncture. Specifically:
a) While the magnetic forces from the magnet 20 radiate to pull the collar 30 (now forward of the magnet), the momentum of the moving collar and outer shaft 50 drive the lancet further to the front 102 of the device 10.
b) The crest 158 continues to be no longer seated with the channel 35 in the collar 30 allowing relative motion between the inner shaft 40 and outer shaft 50.
c) The point 66 of the lancet 60 pierces the imaginary plain of the end 102 of the cap 100 and the cap's opening 104 and the transition begins wherein the lancet goes from a withdrawn position to a piercing position.

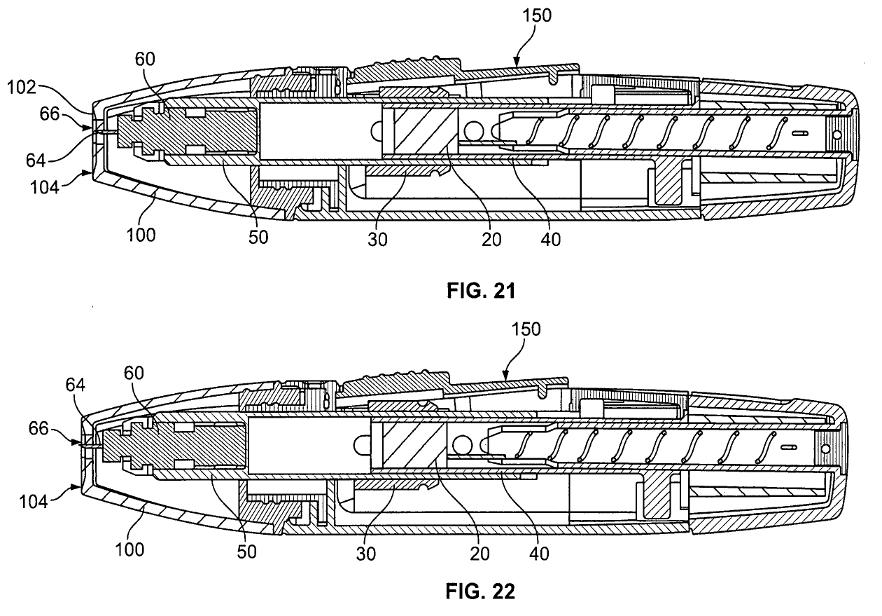

FIG. 22 shows the device 10 and lancet 60 at the fully hyper-extended position or “the piercing position,” namely that position wherein the point 66 of the tip 64 of the lancet 60 is fully extended and the furthest in front of the device 10, cap 100 and cap opening 104. Puncturing of the user is occurring. In particular:
a) While the magnet forces from the magnet 20 radiate to pull or retract the collar 30 (now well forward of the magnet), the momentum of the moving collar and outer shaft 50 drive the lancet to the furthest position in front 102 of the device 10. The advancement of the lancet 60 is can be stopped when the front end 32 of the collar 30 bumps into the dial 170 of the adjuster 160. At such point, all forward motion of the lancet 60 stops immediately. However, ideally, the advancement of the lancet 60 stops due to the properly balanced magnetic force. Such forces are sufficient enough to control the momentum of the collar/lancet. It should be noted that one of the significant advantages of the present device is that it is silent in use because parts do not bump or contact one another during motion. b) The crest 158 continues to be no longer seated with the channel 35 in the collar 30 allowing relative motion between the inner shaft 40 and outer shaft 50.
c) The point 66 of the lancet 60 is well beyond the imaginary plain of the cap's 100 opening 104 and end 102. Transition next begins wherein the lancet goes from the piercing position to a withdrawn position within the cap.

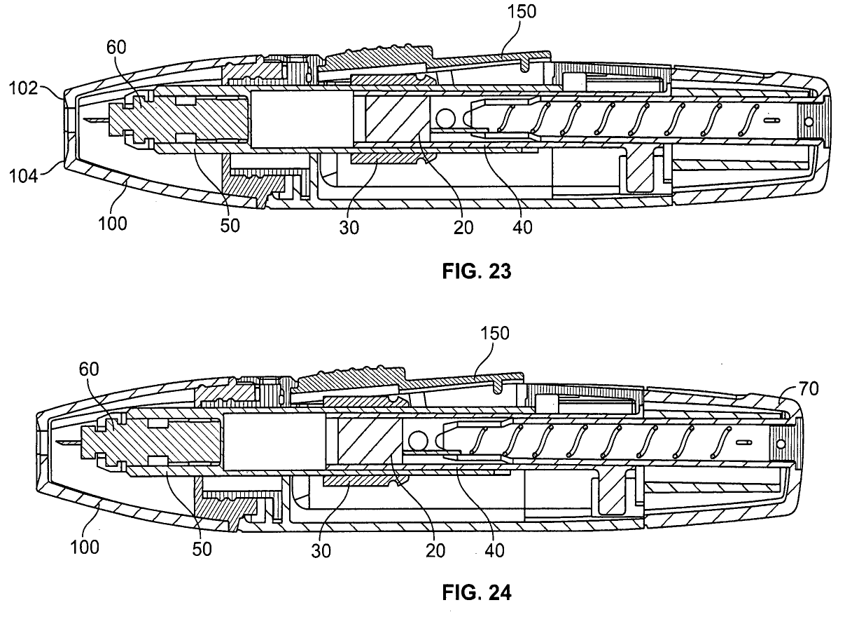

FIG. 23 shows the device 10 and lancet 60 after achieving the piercing position and in a retracting position, wherein the lancet is transitioning back to the steady state condition. Puncturing of the user has occurring and the lancet is withdrawing into the cap. In particular:
a) The magnetic forces from the magnet 20 retract the collar 30 towards the magnet. The lancet 60 having been well forward of the magnet is now drawn towards the magnet. As a result, the point 66 of the tip 64 of the lancet 60, along with the outer shaft 50, move back within the cap 100 and behind cap opening 104 to a withdrawn position.
b) The crest 158 continues not to be seated with the channel 35 in the collar 30 allowing relative motion between the inner shaft 40 and outer shaft 50.
c) The point 66 of the lancet 60 is well behind (as opposed to in front) the imaginary plain of the cap's 100 opening 104. The lancet 60 and its respective tip 64 and point 66 are withdrawing, completely protected by the cap 100 and moving away from the cover's opening 104.

FIG. 24 shows the device 10 and lancet 60 in the steady state condition. The lancet is withdrawn, coming to rest, and the system to equilibrium. As shown:
a) The magnetic forces from the magnet 20 have retracted the collar 30 so that it encircles or is concentric with the magnet. The magnetic forces of the magnet basically hold the collar in this position.
b) The crest 158 remains unseated in the channel 35 in the collar 30 allowing relative motion between the inner shaft 40 and outer shaft 50.
c) The point 66, lancet tip 64 and lancet 60 are well withdrawn, well behind the imaginary plain of the cap's 100 opening 104 and are completely protected by the cap 100.

The device is now in equilibrium and at rest. It will remain in this steady state condition until armed.

Interestingly, in the steady state condition, the device 10 can be dropped or jolted without the lancet 60 or tip 64 extending out of the cap 100. The magnet's 20 magnetic forces hold the collar 30, outer shaft 50 and lancet 60 within their grip. There may be slight relative motion between these parts, e.g., between the inner and outer shafts 40,50, due to external forces, but they should only be slight.

The Magnetic Fields Generated by the Magnet 20

Discussed previously, FIGS. 25 and 26 show density plots of the magnet 20 within the device 10 and the magnetic fields generated thereby in a steady state position (FIG. 26) and at an extreme position (armed position or piercing position)(FIG. 25). FIG. 27 shows a schematic representation of the magnet 20, the magnetic fields and the collar 30 in representative positions. The magnetic field lines MF, or lines of force, associated with the magnet 20 are shown, each line being equal potential. The cylindrical magnet 20, with substantially flat ends 22,23, has a North pole N at one end and a South pole S at the opposed end. This results in magnet field curved lines of flux being generated between both of the poles N,S and around the entire circumference of the magnet. These magnetic field lines and the density plot thereof are shown in FIGS. 25 and 26.

The next figure, FIG. 27, shows the collar in three positions relative to the magnet, namely: a) the collar 30′ is in the steady state position (SS), b) the collar 30″ is in the arming position (AP), and c) the collar 30′″ is in the piercing position (PP). Note, this follows the progression of the collar relative to the magnet—Step 1: The steady state position (SS) to the arming position (AP); Step 2: The arming position (AP) to the piercing position (PP); and Step 3: the piercing position (PP) back to the steady state (SS).

Other Aspects of the Device 10

It should be emphasized that the magnet not only drives the lancet's tip (via the communicating annulet collar) out of the housing or cap, but also back into the housing or cap. Thus, the puncturing process of the present invention involves two steps, both an extension and a withdrawal or retraction of the lancet. This reduces prolonged puncturing and enhances safety of the device and its use.

It should be noted that the above system is described as mechanical. It can, however, incorporate electrical components. Such electrical components should be well recognized by those skilled in the art. For example, arming the device requires physically and mechanically pulling the arming member from the housing. This can also be accomplished by employing gears and an battery driven electrical circuit. In the embodiment described above, activating the device requires physically pressing the button switch. This too can be accomplished by an electronic circuit that uses an electric switch and gears and/or signals to release the collar.

In addition, the embodiment illustrated shows the magnet driving a collar in communications with the lancet. The magnet and collar can be switched so the magnet is in direct communications with the lancet and the magnet moves relative to the collar. Moreover, the magnet and collar are shown to be cylindrical and tubular respectively. It is believed this reduces oscillation or unwanted radial/lateral of the longitudinally moving lancet. Other shapes for the collar and magnet may be employed, such as rectangular, triangular, etc.

Further, the magnetic poles may be reversed in the embodiment shown. Thus, instead of the magnet drawing, pulling and attracting the collar, it can repel and push the collar and visa versa.

While the specific embodiments have been illustrated and described, numerous modifications come to mind without significantly departing from the spirit of the invention and the scope of protection is only limited by the scope of the accompanying claims.

1. A lancing method comprising the steps of:
driving one of a magnetic element and a member capable of being affected by magnetic forces emanating from the magnet in communication with a lancet by the other of the element and the member to pierce a user.

2. The method of claim 1 further including the step of:
withdrawing the driven lancet.

3. A lancing method using a lancing device with a housing and a lancet therein, comprising the steps of:
driving a lancet in communications with one of a magnetic element and a member capable of being affected by magnetic forces emanating from the magnet by the other of the magnetic element and member so a tip of the lancet exits the housing to puncture a user.

4. The method of claim 3 further including the step of:
withdrawing the driven lancet back into the housing also by the other of the magnetic element and the member.

5. The method of claim 3 wherein the step of driving the lancet involves having the one of the magnetic element and the member passing through the other of the magnetic element and the member.

6. A lancing method comprising the steps of:
driving a lancet in communications with one of a magnetic element and a member capable of being attracted and repelled by magnetic forces radiating from the magnet by the other of the element and the member to pierce a user.

7. The method of claim 6 further including the step of:
withdrawing the driven lancet.

8. A lancing method comprising the steps of:
positioning both a magnetic element and a member capable of being affected by magnetic forces emanating from the magnetic element within a housing with a lancet in communications with one of either the magnetic element or the member, the lancet being movable between a withdrawn position wherein the lancet is within the housing and a piercing position wherein the lancet is projecting from the housing and adapted to be movable from a withdrawn position to the piercing position by the movement of one of either the magnetic element or member relative to the other of either the magnetic element or the member;
positioning either the member or the magnetic element to an armed position wherein the magnetic forces from the magnetic element affect the member; and,
releasing the one of either the member or the magnetic element from the armed position permitting movement between the member and magnetic element by at least, in part, the magnetic forces, resulting in the movement of the lancet from a withdrawn position to the piercing position.

9. The method of claim 8 further including the step of:
holding the one of either the member or the magnetic element in the armed position, the lancet being in a withdrawn position.

10. The method of claim 8 further including the step of:
adjusting the lancet for selectively controlling the positioning of the piercing position.

11. The method of claim 8 further including the step of:
adapting the magnetic element and the member so as to permit one to pass through the other and the other to pass around the one.

12. The method of claim 8 further including the step of:
connecting the lancet in communication to the member so that movement of the member results in corresponding movement of the lancet.

13. The method of claim 8 further including the step of:
orienting and configuring the magnetic element and the member within the housing in such manner that in the armed position, the magnetic forces of the magnetic element attract the member to the magnetic element and when the member is released, the member travels towards the magnetic element and past the magnetic element by the momentum of the traveling member resulting in the lancet traveling to the piercing position.

14. The method of claim 8 further including the step of:
orienting and configuring the magnetic element and the member within the housing in such a manner so as to create a steady state position between the withdrawn position and the piercing position wherein the magnetic forces of the magnetic element hold the member concentric therewith and the lancet is within the housing.

15. The method of claim 8 further including the step of:
orienting and configuring the magnetic element and the member within the housing such that in the armed position, the magnetic forces of the magnetic element attract the member to the magnetic element and when the member is released, the member travels towards the magnetic element, through the steady state position concentric with the magnet, past the magnetic element by the momentum of the traveling member and back to the steady state position resulting in the lancet traveling to the piercing position and back to a position within the housing.

16. The method of claim 8 further including the steps of:
fixing the magnetic element within an inner shaft; and
fixing the member around an outer shaft;
moving the outer shaft relative to the inner shaft.

17. The method of claim 8 further including the step of:
releasably connecting the lancet to the outer shaft.

18. The method of claim 8 further including the step of
releasably connecting an end cap to the housing.

19. The method of claim 8 wherein the member is selectively held in the armed position or released from the armed position by a switch.

20. The method of claim 19 wherein the switch is selectively engaging or disengaging a the member, the member being held in the armed position and the lancet is in the withdrawn position when the switch engages the member and the member and lancet being free to move from the armed and withdrawn positions when the switch is disengaged from the member.

21. The method of claim 8 wherein the step of positioning either the member or the magnetic element to an armed position wherein the magnetic forces from the magnetic element affect the member involves moving an arming member which mechanically moves the collar from the steady state position to the armed position.

22. The method of claim 21 further including the step of:
holding the arming member to the housing by at least one spring.
